(19) United States
(12) Patent Application Publication
(10) Pub. No.: US 2023/0330041 A1
(43) Pub. Date: Oct. 19, 2023

(54) APPLICATION OF TRIS (HYDROXYMETHYL) AMINOMETHANE PHARMACEUTICALLY ACCEPTABLE SALT TO DRUGS FOR TREATING HYPERURICEMIA

(71) Applicant: BEIJING CHANGSHENG PHARMACEUTICAL TECHNOLOGY CO., LTD., Beijing (CN)

(72) Inventors: Guowen LI, Beijing (CN); Xianhua CHEN, Beijing (CN)

(21) Appl. No.: 18/043,068

(22) PCT Filed: Aug. 20, 2021
(86) PCT No.: PCT/CN2021/113858

§ 371 Date: Feb. 27, 2023

(30) Foreign Application Priority Data
Aug. 27, 2020 (CN) 202010874220.2
Jun. 11, 2021 (CN) 202110651157.0

(51) Int. Cl. A61K 31/133; A61P 19/06

(52) U.S. Cl. CPC A61K 31/133; A61P 19/06

(57) ABSTRACT

A use of a tris (hydroxymethyl) aminomethane pharmaceutically acceptable salt or a composition formed by tris (hydroxymethyl) aminomethane and an inorganic acid or an organic acid in the preparation of drugs for treating or preventing hyperuricemia, gout, acute and chronic arthritis, gouty nephropathy, uric acid nephrolithiasis and related diseases. A use of tris (hydroxymethyl) aminomethane in the preparation of topical pharmaceutical compositions for preventing or treating gout and gouty arthritis by topical and transdermal administration.

### CROSS-REFERENCE TO RELATED APPLICATIONS

This application claims priority to and the benefit of the Chinese Pat. Application No. 202010874220.2 filed on Aug. 27, 2020 and the Chinese Pat. Application No. 202110651157.0 filed on Jun. 11, 2021, the entire contents of which are incorporated herein by reference.

### FIELD

The present disclosure relates to the field of drug application, and specifically relates to use of a pharmaceutically acceptable salt of trimethylol aminomethane in the manufacture of a medicament for treating or preventing hyperuricemia, gout, acute and chronic arthritis, gouty nephropathy and uric acid kidney stone and related diseases, and more specific to use of a trimethylol aminomethane hydrochloride or salt solutions in the manufacture of a medicament for preventing or treating hyperuricemia, gout, acute and chronic arthritis, gouty nephropathy and uric acid kidney stone and related diseases.

### BACKGROUND

“Hyperuricemia” refers to any one of a disease, symptom or condition that presents an over-high uric acid content in serum such as greater than 6.8 mg/dL for men and greater than 6.0 mg/dL for women. Related diseases include but are not limited to gout, gouty arthritis, cerebral stroke, ischemic heart disease, renal dysfunction, uremia, lithangiuria, gouty nephropathy, kidney stone or chronic kidney disease (CKD).

Gout is a group of diseases caused by purine metabolism disorder, which is characterized by hyperuricemia and repeated attacks of acute gouty arthritis, tophus deposition, chronic gouty arthritis and joint deformity caused by hyperuricemia. It often involves the kidney, causing chronic interstitial nephritis and uric acid kidney stone formation. The disease may be divided into two categories, primary disease and secondary disease, where a few of the primary diseases is caused by enzyme deficiency, often accompanied by hyperlipidemia, obesity, diabetes, hypertension, arteriosclerosis and coronary heart disease, and the secondary disease may be caused by kidney disease, blood disease and drugs and other reasons. Numerous scientific studies have established that uric acid crystals cause gout.

According to the theory of dissolution and crystallization of a simple compound in solution, the solute should crystallize out if it is higher than the solubility. The chemical solubility of uric acid in blood is 420 µmol/L (7 mg/dL). If a uric acid concentration in the blood exceeds this critical point, uric acid crystals should be formed in the blood. However, uric acid crystals have never been found in the plasma of patients with hyperuricemia. The highest blood uric acid value reported clinically is 1185 µmol/L, and 765 µmol/L of the blood uric acid higher than the solubility critical point can be dissolved in the blood, because uric acid is bound to lipoprotein or red blood cells or other substances in the blood, and the binding between them is hydrogen bonding, instead of chemical bonding, as their binding force is very weak (reversible). After uric acid binds to a lipoprotein and the like in the blood, it will no longer binds to another uric acid molecule (crystallization), that is why 85% of people with hyperuricemia have no gout symptoms.

Although 85% of patients with hyperuricemia have no gout symptoms, they suffer from various diseases related to hyperuricemia.

A large number of studies have proved that hyperuricemia closely relates to hyperlipidemia, renal impairment, obesity, hypertension and diabetes, and promotes each other. Uric acid is positively correlated with low density lipoprotein and blood glucose.

For example, uric acid binds to low density lipoprotein cholesterol (LDL-C) and then promotes its oxidation to oxidized low-density lipoprotein cholesterol (Ox-LDL-C). Ox-LDL and Ox-LDL-C deposit on the inner wall of blood vessels, leading to atherosclerosis and making patients with high uric acid suffer from cardiovascular and cerebrovascular diseases such as atherosclerosis.

That is, high uric acid is harmful to the human body in two ways, i) crystallization of the uric acid harms the human body; and ii) the combination of the uric acid with blood lipid, blood glucose, enzyme protein and other macromolecular substances in blood causes the negative influence on the blood lipid, blood glucose and enzyme protein activity.

In the related art, there is no drug that can directly solve the problem of the combination of uric acid with blood lipid, blood glucose, enzyme protein and other macromolecular substances in blood.

At present, there are three main methods for treating hyperuricemia: a. reducing intake; b. inhibiting endogenous synthesis; and c. promoting renal excretion. However, these three methods show preventive and treatment effects with certain limitations.

Hyperuricemia, gout and related diseases seriously affect the quality of life, labor capacity, and even endanger life.

Therefore, there is an urgent need for the prevention and treatment of hyperuricemia.

### SUMMARY

In a first aspect, the present disclosure provides in embodiments use of a pharmaceutically acceptable salt of trimethylol aminomethane in the manufacture of a composition for preventing or treating hyperuricemia.

In some embodiments of the first aspect, an acid forming the salt with trimethylol aminomethane comprises an organic acid and an inorganic acid, the inorganic acid or organic acid is selected from a group consisting of a hydrochloric acid, lysine, ascorbic acid, acetic acid, formic acid, phosphoric acid, sulfuric acid, maleic acid or butanedisulfonic acid, so as to form the salt with trimethylol aminomethane or form a compound via hydrogen bonding, preferably the pharmaceutically acceptable salt of trimethylol aminomethane is a trimethylol aminomethane hydrochloride.

In other embodiments of the first aspect, the hyperuricemia is gout, gouty nephropathy, gouty arthritis and uric acid kidney stone, preferably the hyperuricemia is a disease with an over-high uric acid content greater than 420 µmol/L for men and greater than 360 µmol/L for women, gout, gouty arthritis, cerebral stroke, ischemic heart disease, renal dysfunction, uremia, lithangiuria, urate nephropathy, kidney stone or chronic kidney disease (CKD).

In still other embodiments of the first aspect, the pharmaceutically acceptable salt of trimethylol aminomethane exhibits the following effects: preventing crystallization of a uric acid, dissolving a crystal of the uric acid and reducing a combination of the uric acid with a macromolecular substance such as blood fat or blood sugar so as to reduce the negative influence on the blood fat and the blood sugar.

In yet other embodiments of the first aspect, the composition comprises a therapeutically effective amount of the pharmaceutically acceptable salt of trimethylol aminomethane and a food acceptable or pharmaceutically acceptable excipients.

In yet other embodiments of the first aspect, the composition is selected from a pharmaceutical composition, a feedstuff composition, a food composition or a health-care composition, optionally the pharmaceutical composition is a human medicament or a poultry medicament.

In yet other embodiments of the first aspect, the pharmaceutical composition is formulated in the form of oral liquids, tablets, capsules, granules, powders, pills, syrups, injections, drops, inhalants, gel ointments, sprays, dialysis liquids or dialysis powders, optionally, the composition is administered via infusion, orally, smearing, dressing or dialysis.

In yet other embodiments of the first aspect, the composition is formulated as an aqueous solution with a pH value of 5.0 to 11.0, preferably an aqueous solution with a pH value of 7.4 ± 0.5.

In yet other embodiments of the first aspect, a concentration of the pharmaceutically acceptable salt of trimethylol aminomethane in the composition is 0.6±0.4 mol/L, preferably 0.3 mol/L, more preferably 0.6 mol/L.

In a second aspect, the present disclosure provides in embodiments a pharmaceutically acceptable salt of trimethylol aminomethane or a composition comprising the same for use in prevention or treatment of hyperuricemia, wherein the prevention or treatment is achieved by forming a uric acid - trimethylol aminomethane complex via hydrogen bonding between the pharmaceutically acceptable salt of trimethylol aminomethane and a uric acid,

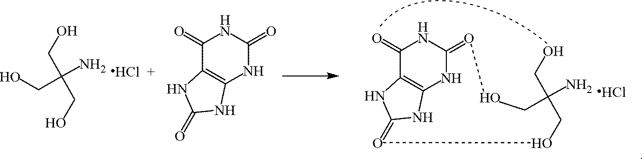

In some embodiments of the second aspect, an acid forming the salt with trimethylol aminomethane comprises an organic acid and an inorganic acid, the inorganic acid or organic acid is selected from a group consisting of a hydrochloric acid, lysine, ascorbic acid, acetic acid, formic acid, phosphoric acid, sulfuric acid, maleic acid or butanedisulfonic acid, so as to form the salt with trimethylol aminomethane or form a compound via hydrogen bonding, preferably the pharmaceutically acceptable salt of trimethylol aminomethane is a trimethylol aminomethane hydrochloride.

In other embodiments of the second aspect, the hyperuricemia is gout, gouty nephropathy, gouty arthritis and uric acid kidney stone, preferably the hyperuricemia is a disease with an over-high uric acid content greater than 420 µmol/L for men and greater than 360 µmol/L for women, gout, gouty arthritis, cerebral stroke, ischemic heart disease, renal dysfunction, uremia, lithangiuria, urate nephropathy, kidney stone or chronic kidney disease (CKD).

In still other embodiments of the second aspect, the pharmaceutically acceptable salt of trimethylol aminomethane exhibits the following effects: preventing crystallization of a uric acid, dissolving a crystal of the uric acid and reducing a combination of the uric acid with a macromolecular substance such as blood fat or blood sugar so as to reduce the negative influence on the blood fat and the blood sugar.

In yet other embodiments of the second aspect, the composition comprises a therapeutically effective amount of the pharmaceutically acceptable salt of trimethylol aminomethane and a food acceptable or pharmaceutically acceptable excipients.

In yet other embodiments of the second aspect, the composition is selected from a pharmaceutical composition, a feedstuff composition, a food composition or a health-care composition, optionally the pharmaceutical composition is a human medicament or a poultry medicament.

In yet other embodiments of the second aspect, the pharmaceutical composition is formulated in the form of oral liquids, tablets, capsules, granules, powders, pills, syrups, injections, drops, inhalants, gel ointments, sprays, dialysis liquids or dialysis powders, optionally, the composition is administered via infusion, orally, smearing, dressing or dialysis.

In yet other embodiments of the second aspect, the composition is formulated as an aqueous solution with a pH value of 5.0 to 11.0, preferably an aqueous solution with a pH value of 7.4 ± 0.5.

In yet other embodiments of the second aspect, a concentration of the pharmaceutically acceptable salt of trimethylol aminomethane in the composition is 0.6±0.4 mol/L, preferably 0.3 mol/L, more preferably 0.6 mol/L.

In a third aspect, the present disclosure provides in embodiments a method of prevention or treatment of hyperuricemia, comprising administering a therapeutically effective amount of a pharmaceutically acceptable salt of trimethylol aminomethane or a composition comprising the same to a subject in need thereof, wherein the prevention or treatment is achieved by forming a uric acid - trimethylol aminomethane complex via hydrogen bonding between trimethylol aminomethane and a uric acid.

In other embodiments of the third aspect, an acid forming the salt with trimethylol aminomethane comprises an organic acid and an inorganic acid, the inorganic acid or organic acid is selected from a group consisting of a hydrochloric acid, lysine, ascorbic acid, acetic acid, formic acid, phosphoric acid, sulfuric acid, maleic acid or butanedisulfonic acid, so as to form the salt with trimethylol aminomethane or form a compound via hydrogen bonding, preferably the pharmaceutically acceptable salt of trimethylol aminomethane is a trimethylol aminomethane hydrochloride.

In still other embodiments of the third aspect, the hyperuricemia is gout, gouty nephropathy, gouty arthritis and uric acid kidney stone, preferably the hyperuricemia is a disease with an over-high uric acid content greater than 420 µmol/L for men and greater than 360 µmol/L for women, gout, gouty arthritis, cerebral stroke, ischemic heart disease, renal dysfunction, uremia, lithangiuria, urate nephropathy, kidney stone or chronic kidney disease (CKD).

In yet other embodiments of the third aspect, the pharmaceutically acceptable salt of trimethylol aminomethane exhibits the following effects: preventing crystallization of a uric acid, dissolving a crystal of the uric acid and reducing a combination of the uric acid with a macromolecular substance such as blood fat or blood sugar so as to reduce the negative influence on the blood fat and the blood sugar.

In yet other embodiments of the third aspect, the composition comprises a therapeutically effective amount of the pharmaceutically acceptable salt of trimethylol aminomethane and a food acceptable or pharmaceutically acceptable excipients.

In yet other embodiments of the third aspect, the composition is selected from a pharmaceutical composition, a feedstuff composition, a food composition or a health-care composition, optionally the pharmaceutical composition is a human medicament or a poultry medicament.

In yet other embodiments of the third aspect, the subject comprises a mammal or a non-mammal, preferably, the mammal is human, preferably, the non-mammal is poultry, more preferably, the poultry is a chicken.

In yet other embodiments of the third aspect, the pharmaceutically acceptable salt of trimethylol aminomethane or a composition comprising the same is administered to the subject via infusion, orally, smearing, dressing or dialysis.

In yet other embodiments of the third aspect, the amount for administration is 0.1 g/kg to 10 g/kg body weight per day, preferably 1.7 g/kg body weight per day, more preferably 3.4 g/kg body weight per day; or the amount for administration is 0.5 ml/kg/dose to 3 ml/kg/dose based on 0.3 mol/L or 0.6 mol/L the pharmaceutically acceptable salt of trimethylol aminomethane in solutions, respectively.

In yet other embodiments of the third aspect, the composition is formulated as an aqueous solution with a pH value of 5.0 to 11.0, preferably an aqueous solution with a pH value of 7.4 ± 0.5.

In a fourth aspect, the present disclosure provides in embodiments use of trimethylol aminomethane in the manufacture of a topical pharmaceutical composition for preventing or treating gout or gouty arthritis via topical transdermal administration.

In some embodiments of the fourth aspect, trimethylol aminomethane is in the form of creams, ointments, gels, transdermal preparations, foams, sprays, lotions, solutions, emulsions or suspensions, preferably gels.

In a fifth aspect, the present disclosure provides in embodiments a method of prevention or treatment of gout or gouty arthritis, comprising administering a therapeutically effective amount of trimethylol aminomethane or a topical pharmaceutical composition comprising the same to a subject in need thereof via topical transdermal administration, wherein the prevention or treatment is achieved by forming a uric acid - trimethylol aminomethane complex via hydrogen bonding between trimethylol aminomethane and a uric acid.

In a sixth aspect, the present disclosure provides in embodiments trimethylol aminomethane or a topical pharmaceutical composition comprising the same for use in prevention or treatment of gout or gouty arthritis via topical transdermal administration, wherein the prevention or treatment is achieved by forming a uric acid - trimethylol aminomethane complex via hydrogen bonding between trimethylol aminomethane and a uric acid,

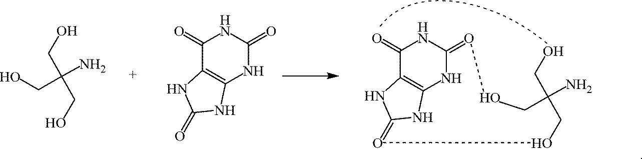

In a seventh aspect, the present disclosure provides in embodiments use of trimethylol aminomethane in the manufacture of a veterinary composition or a feedstuff composition for preventing or treating hyperuricemia, gout or gouty arthritis in poultry.

In an eighth aspect, the present disclosure provides in embodiments a method of prevention or treatment of hyperuricemia, gout or gouty arthritis in poultry, comprising administering a therapeutically effective amount of trimethylol aminomethane or a composition comprising the same to a poultry subject in need thereof, wherein the prevention or treatment is achieved by forming a uric acid - trimethylol aminomethane complex via hydrogen bonding between trimethylol aminomethane and a uric acid.

In a ninth aspect, the present disclosure provides in embodiments trimethylol aminomethane for use in prevention or treatment of hyperuricemia, gout or gouty arthritis in poultry via a veterinary composition or a feedstuff composition, wherein the prevention or treatment is achieved by forming a uric acid - trimethylol aminomethane complex via hydrogen bonding between trimethylol aminomethane and a uric acid,

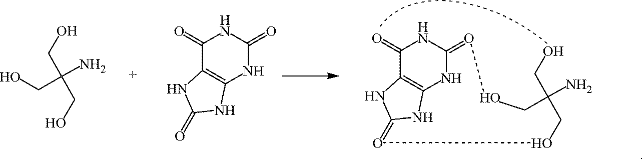

### BRIEF DESCRIPTION OF THE DRAWINGS

FIG. 1 is an infrared detection spectrum for a uric acid - trimethylol aminomethane complex.

FIG. 2 shows effect of a trimethylol aminomethane hydrochloride (also named as Changsheng No. 1) on renal uric acid crystal deposition, where an arrow indicates the crystal deposition of uric acid in the kidney, and A is a normal control group, B is a model control group, C is a group treated with 0.85 g/kg of Changsheng No. 1, D is a group treated with 1.70 g/kg of Changsheng No. 1, and E is a group treated with 3.40 g/kg of Changsheng No. 1.

FIG. 3 shows effect of the trimethylol aminomethane hydrochloride (also named as Changsheng No. 1) on kidney weight, comparing with the model control, where P<0.05 and P<0.001.

FIG. 4 shows effect of the trimethylol aminomethane hydrochloride (also named as Changsheng No. 1) on renal coefficient, comparing with the model control, where P<0.05 and P<0.001.

FIG. 5 shows observed results of uric acid crystallization in rat kidneys, where the kidney in the group treated with 3.40 g/kg of Changsheng No. 1 presents no difference from that of the normal control group.

### DETAILED DESCRIPTION OF EMBODIMENTS

The embodiments of the present disclosure are described in detail below, which are exemplary and only for explaining the present disclosure, but not for limiting the present disclosure.

The present application is made based on the discovery of the following problems and facts by the inventor.

In order to solve the problem of the harm of uric acid to human body, the molecular structure and physicochemical properties of uric acid should be analyzed to seek the causes of uric acid crystallization, to find new drugs with clear action mechanism that can effectively solve the problem of uric acid crystallization and the influence of uric acid on macromolecular proteases and the like.

Finding new properties of known compounds and then making drugs based on the discovered new properties is one of the common methods for developing new drugs.

There is a known compound, tromethamine, with Chinese alias of trimethylol aminomethane.

CAS number of it is 77-86-1, and the molecular formula is C₄H₁₁NO₃, with a molecular weight of 121.135.

There is an amino group in tromethamine, which has strong alkalinity and can neutralize H+ in solutions and correct metabolic acidemia. It is a commonly used acid-base balance drug for treating blood acidosis, and is used for acute respiratory or metabolic acidosis and alkalization of urine to solve the problem of human urine acidity (low pH). However, there are varieties of serious adverse reactions as tromethamine has strong alkalinity, strong irritation and side effects.

According to the general knowledge of biology and enzymology, the main reasons for the serious adverse reactions of tromethamine injection are as follows: the pH value of the trometamol injection is 10.5~11.5 of which the alkalinity is too strong, and when a large amount of alkali liquor enters the human body, it will make many key enzyme proteins in the human body lose activity or denature, thereby causing a series of adverse reactions.

In the related art, known effects of tromethamine are only limited to that one amino group of it has strong alkalinity, which can neutralize H+ in solutions and correct metabolic academia; and tromethamine is an acid-base balance drug, and is used for acute respiratory or metabolic acidosis and alkalization of urine to solve the problem of human urine acidity (low pH).

There is a large number of the existing literature attempting to use tromethamine (trimethylol aminomethane) to treat gouty urinary uric acid stone disease by alkalizing urine and promoting the dissolution of urinary uric acid stone, by utilizing the alkalinity of its amino group combining with H+. That is sometimes referred to simply as “trimethylol aminomethane for the treatment of gouty urinary uric acid stone disease,” and more simply as “trimethylol aminomethane for the treatment of gout and hyperuricemia.” In fact, these simplifications are not scientific. Medical terminology must be scientific and rigorous. For example, piperacillin has the effect of killing pseudomonas aeruginosa, thereby being capable of treating bacterial infectious diseases caused by pseudomonas aeruginosa infection. It cannot be expressed in general that “piperacillin can treat bacterial infectious diseases”, because it only treats diseases of pseudomonas aeruginosa infections, but cannot treat other bacterial infections. General and imprecise misstatements will expand the efficacy and scope of application of drugs.

Therefore, it is not scientific and is incorrect to simplify the treatment of urinary uric acid stones by alkalizing urine as “the treatment of gout and hyperuricemia with trimethylol aminomethane”.

The technical solutions for treating gout and hyperuricemia with trimethylol aminomethane in the existing literature actually solve the problems only limited to utilizing the alkalinity of its amino group to combine with H+ and thus alkalizing urine, to promote dissolutions of uric acid crystals or stones.

The technical solution of the disclosure for treating gout and hyperuricemia with trimethylol aminomethane actually solve the following problems: the problem of uric acid molecules combining with each other through hydrogen bonding easily (crystallized); and the problem of uric acid combining with blood sugar and other macromolecular substances to cause a variety of diseases. For example, due to the combination of uric acid and low density lipoprotein, low density lipoprotein is oxidized into oxidized low density lipoprotein, and then deposited thereby forming thrombus, causing cardiovascular and cerebrovascular diseases, atherosclerosis and other diseases related to high uric acid. A reaction equation known as for “performance” and “therapeutic mechanism” of trimethylol aminomethane (tromethamine) in the existing literature is as follows:

$${\text{C}}_{\text{4}} {\text{H}}_{\text{11}} {\text{NO}}_{\text{3}} ⋅ {\text{H}}_{\text{2}} {\text{O+H}}^{\text{+}} {\text{=C}}_{\text{4}} {\text{H}}_{\text{11}} {\text{NO}}_{\text{3}} {\text{H}}^{\text{+}} {\text{+H}}_{\text{2}} \text{O} \text{.}$$

In the related art, not all drugs, after salification, will still have their original efficacy, and can still treat the original disease.

If a compound is used to treat disease (efficacy) with its alkalinity, it cannot be salified, because if salified with an acid, it will lose its original efficacy and cannot treat the original disease.

In the prior art, it is the alkalinity of tromethamine (trimethylol aminomethane) that is used to treat diseases (efficacy), and thus it cannot be prepared into salt, and if salified with an acid, it will lose its original efficacy and cannot treat the original disease.

In the prior art, the efficacy of tromethamine to alkalize urine and promote dissolution of urinary stone is achieved by utilizing the alkalinity of its amino group combining with H+ thereby improving the pH value of urine to alkalize urine.

If tromethamine is salified with a hydrochloric acid, a tromethamine hydrochloride could not combine with H+ anymore, and the salified tromethamine would loss the original efficacy of alkalizing urine. A reaction equation for salifying with tromethamine is as follows:

$${\text{C}}_{\text{4}} {\text{H}}_{\text{11}} {\text{NO}}_{\text{3}} {\text{+HCl=C}}_{\text{4}} {\text{H}}_{\text{11}} {\text{NO}}_{\text{3}} {\text{H}}^{\text{+}} {\text{Cl}}^{\text{-}} .$$

As tromethamine is too alkaline to be taken orally, it has been suggested to prepare tromethamine into tromethamine lactate or tromethamine acetate, and to take the tromethamine lactate or tromethamine acetate to alkalize urine, thereby promoting the dissolution of uric acid stones in the urinary system and treating gouty urinary uric acid lithiasis. However, anyone with a little chemistry knowledge will realize that, such a tromethamine lactate or acetate has combined with one H+ already, and thus could not combine with more H+ further, so it cannot alkalize urine to treat gouty urinary uric acid lithiasis.

Therefore, tromethamine, a drug for alkalizing urine, cannot be prepared into salt to alkalize urine and to treat uric acid crystallization in order to improve its convenience of use like most drugs.

Thus no one would try to prepare a salt of tromethamine (trimethylol aminomethane hydrochloride), where tromethamine is a drug that alkalizes urine.

On the market, the common chemical reagent “trimethylol aminomethane hydrochloride”, which coexists with “trimethylol aminomethane” and has a CAS No. of 1185-53-1 and a molecular formula of C₄H₁₂ClNO₃ with a molecular weight of 156.589, is an existing, known and commonly used compound on the market that coexists with “trimethylol aminomethane” and is a chemical reagent commonly used in many biochemical experiments as well.

In the prior art, the known performance of trimethylol aminomethane hydrochloride is only limited to be taken as a commonly used chemical reagent for biochemical tests or as an intermediate in organic synthesis. The amino group in trimethylol aminomethane hydrochloride has combined with one H+ already, and thus could not alkalize urine to treat urinary uric acid stone, let alone treat hyperuricemia.

There is no disclosure about a hydrochloride or salt solutions of trimethylol aminomethane in the manufacture of a medicament for preventing or treating hyperuricemia, gout, acute and chronic arthritis, gouty nephropathy and uric acid kidney stone and related diseases.

There is no disclosure about a pharmaceutically acceptable salt of trimethylol aminomethane or a composition formed by trimethylol aminomethane and an organic acid or an inorganic acid in the manufacture of a medicament for preventing or treating hyperuricemia, gout, acute and chronic arthritis, gouty nephropathy and uric acid kidney stone and related diseases.

In addition, the prior art only utilizes the alkalinity of amino group in trimethylol aminomethane to improve the pH value of urine, and attempts to alkalizing urine to promote the dissolution of urinary uric acid stone to treat urinary uric acid stone disease.

Since that the pH value of the blood of the human body is kept stable, trimethylol aminomethane has very limited effects on the adjustment of the pH value of the blood, and on the dissolution of uric acid crystals, and the alkalinity of trimethylol aminomethane has a large toxic side effect on the activity of various biological enzymes in the blood, so the prior art is only limited to injection or oral administration for treating metabolic acidosis by neutralizing acidity and for treating urinary uric acid stone by alkalizing urine, with acid-base neutralization, but no one attempts to treat diseases such as gouty arthritis via topical transdermal administration.

The present disclosure provides in embodiments use of a pharmaceutically acceptable salt of trimethylol aminomethane or a composition formed by trimethylol aminomethane and an organic acid or an inorganic acid in the manufacture of a medicament for preventing or treating hyperuricemia, gout, acute and chronic arthritis, gouty nephropathy and uric acid kidney stone and related diseases. Particularly, the present disclosure provides in embodiments use of a trimethylol aminomethane hydrochloride or its salt solutions in the manufacture of a medicament for preventing or treating hyperuricemia, gout, acute and chronic arthritis, gouty nephropathy and uric acid kidney stone and related diseases.

Finding new properties of known compounds and then making drugs based on the discovered new properties is one of the common methods for developing new drugs.

In processes of chemical experiments, the inventor accidentally discovered a new property that three hydroxyl groups in the molecules of the known compounds, namely trimethylol aminomethane hydrochloride and trimethylol aminomethane, could bind with three oxygen atoms of a uric acid, and according to this new property drugs for treating hyperuricemia and related diseases were prepared. The drug directly combines with uric acid in blood, and thus preventing crystallization of uric acid, dissolving uric acid crystals and reducing the combination of the uric acid with a macromolecular substance such as blood fat or blood sugar to reduce the negative influence on the blood fat and the blood sugar. The present disclosure is an invention that finds new pharmaceutical use of known compounds with new properties.

On Mar. 21, 2020, the inventor unexpectedly found that: by adding 300 mg of uric acid into 100 ml of 0.6 mol/L (pH 7.4) of a trimethylol aminomethane hydrochloride solution and stirring for 10 minutes, the uric acid were gradually dissolved, and the solution became clear; with stirring continually, then milky flocculent precipitate was generate; and with vacuum filtration and drying, 480 mg of uric acid - trimethylol aminomethane complex in white lump were obtained. As the presence of “trimethylol aminomethane” and “trimethylol aminomethane hydrochloride” in the trimethylol aminomethane hydrochloride solution with pH 7.4, the products included “uric acid · trimethylol aminomethane” and “uric acid · trimethylol aminomethane hydrochloride”.

The reaction equations are as follows:

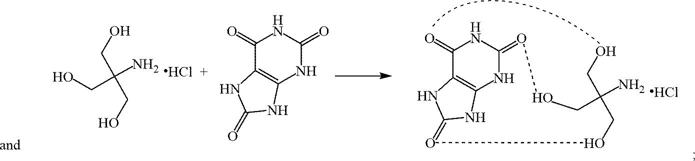

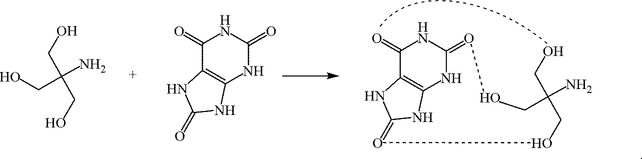

where the “trimethylol aminomethane hydrochloride” bond with uric acid to form a “uric acid · trimethylol aminomethane hydrochloride” complex, and the “trimethylol aminomethane” bond with uric acid to form a “uric acid · trimethylol aminomethane” complex, and the principle for these are the three hydroxyl groups of trimethylol aminomethane binding with three oxygen atoms of a uric acid via hydrogen bonds.

That is, the inventor surprisingly found that: a. the “trimethylol aminomethane hydrochloride” can bind with uric acid by via hydrogen binding to form the “uric acid · trimethylol aminomethane hydrochloride” complex; b. the “trimethylol aminomethane” can bind with uric acid by via hydrogen binding to form the “uric acid · trimethylol aminomethane” complex; c. after binding to the “trimethylol aminomethane hydrochloride” or “trimethylol aminomethane”, the uric acid will no longer bind to another uric acid molecule (crystallization); and d. after binding to the “trimethylol aminomethane hydrochloride” or “trimethylol aminomethane”, the uric acid will no longer bind to macromolecular substances in the blood such as blood fat, blood sugar and protease, and thus will not influence biological activity of the macromolecular substances in the blood such as blood fat, blood sugar and protease.

The present disclosure finds a new property that three hydroxyl groups in the molecules of commonly used compounds, namely trimethylol aminomethane hydrochloride and trimethylol aminomethane, could bind with three oxygen atoms of uric acid, and prepares drugs for treating hyperuricemia and related diseases according to this new property.

The present disclosure in fact, by the strictest definition, finds a. the second medical use of the compound “trimethylol aminomethane hydrochloride” for treating a variety of diseases caused by the combination of uric acid and macromolecular substances such as blood fat and blood sugar, where specific diseases are, for example, cardiovascular and cerebrovascular diseases, atherosclerosis and other diseases related to high uric acid caused by the combination of uric acid and low density lipoprotein, where the low density lipoprotein is oxidized into oxidized low density lipoprotein, and then oxidized low density lipoprotein cholesterin is deposited thereby forming thrombus; and b. the first medical use of the known compound “trimethylol aminomethane hydrochloride”.

In the prior art, the “trimethylol aminomethane hydrochloride” is a chemical reagent and has never been used as a drug.

The present disclosure finds the “new property” of the “trimethylol aminomethane hydrochloride” to bind with uric acid, and this property was used to make drugs that bind to uric acid in the blood to treat hyperuricemia.

Like inventions of most compound drugs, the present disclosure discovered the new property that the trimethylol aminomethane salt as a salt compound can combine with uric acid during a chemical test process firstly, and then provides a medical-use invention of preparing this compound into drugs by utilizing the new property, based on the discovery of the new property of the product.

Comparisons between the present disclosure and the prior art are as follows.

The prior art 1: in the prior art known effects of the known compound tromethamine (trimethylol aminomethane) are only limited to that one amino group of it has strong alkalinity, which can neutralize H+ in solutions and correct metabolic academia; and tromethamine is an acid-base balance drug, used for acute respiratory or metabolic acidosis, and there are also attempts to alkalize urine to solve the problem of human urine acidity (low pH), to treat gouty urinary uric acid stone disease by alkalizing urine and promoting the dissolution of urinary uric acid stone.

The reaction equation for the alkalization of urine by tromethamine (trimethylol aminomethane) is as follows:

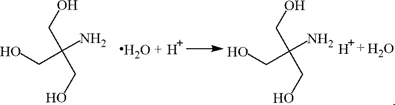

That is, the prior art is only limited to injection or oral administration for treating metabolic acidosis by neutralizing acidity and for treating urinary uric acid stone by alkalizing urine, with acid-base neutralization.

The present disclosure discovers the new property of the “trimethylol aminomethane” that it can bind with uric acid via hydrogen binding to form the “uric acid · trimethylol aminomethane” complex, and thus breaks through the limitation that the prior art only treats urinary calculi by alkalizing urine by utilizing the alkalinity of amino groups of trimethylol aminomethane to neutralize H+. The trimethylol aminomethane can directly combine with uric acid molecules in blood to form the uric acid - trimethylol aminomethane complex, thereby preventing crystallization of uric acid, dissolving crystals of the uric acid and reducing the combination of the uric acid with macromolecular substances such as blood fat or blood sugar to reduce the negative influence on the blood fat and the blood sugar. The present disclosure could be widely used for various diseases caused by uric acid crystallization and high uric acid with high uric acid value, for example, can relieve cardiovascular and cerebrovascular diseases, atherosclerosis and other diseases related to high uric acid by avoiding thrombus formed by deposition of oxidized low-density lipoprotein and oxidized low-density lipoprotein cholesterol caused by the combination with uric acid.

Adaptation diseases of trimethylol aminomethane in the prior art are limited to the treatment of metabolic acidosis and the treatment of urinary uric acid stones by alkalizing urine.

Adaptation diseases of trimethylol aminomethane in embodiments of the present disclosure are all of diseases with an over-high uric acid content in serum such as greater than 6.8 mg/dL for men and greater than 6.0 mg/dL for women, including gout, gouty arthritis, cerebral stroke, ischemic heart disease, renal dysfunction, uremia, lithangiuria, gouty nephropathy, kidney stone or chronic kidney disease (CKD), which are related to hyperuricemia. Specific diseases may be, for example, toe joint gout pain, cardiovascular and cerebrovascular diseases and the like caused by the combination of uric acid molecules and lipoproteins.

The range of the adaptation diseases in embodiments of the present disclosure is much wider than that of the prior art, and is suitable for many new adaptation diseases, such as toe joint gout pain, cardiovascular and cerebrovascular diseases caused by the combination of uric acid molecules and lipoproteins and the like, as well as cardiovascular and cerebrovascular diseases, atherosclerosis and other diseases related to high uric acid caused by the combination of uric acid and low density lipoprotein, where the low density lipoprotein is oxidized into oxidized low density lipoprotein, and then oxidized low density lipoprotein cholesterin is deposited thereby forming thrombus. The present disclosure has significant progress.

In the prior art 2, the known performance of trimethylol aminomethane hydrochloride is only limited to be taken as a commonly used chemical reagent for biochemical tests or as an intermediate in organic synthesis. The amino group in trimethylol aminomethane hydrochloride has combined with one H+ already, and thus could not alkalize urine to treat urinary uric acid stone, let alone treat hyperuricemia.

The present disclosure discovers the new property of the “trimethylol aminomethane hydrochloride” that it can bind with uric acid via hydrogen binding to form the “uric acid · trimethylol aminomethane hydrochloride” complex, and thus breaks through the limitations that the prior art only takes it as a commonly used chemical reagent for biochemical tests or as an intermediate in organic synthesis, and breaks through the limitations that the trimethylol aminomethane hydrochloride could not alkalize urine to treat urinary uric acid stone and could not treat hyperuricemia. The trimethylol aminomethane hydrochloride can directly combine with uric acid molecules in blood to form the uric acid · trimethylol aminomethane hydrochloride complex, thereby preventing crystallization of uric acid, dissolving crystals of the uric acid and reducing the combination of the uric acid with macromolecular substances such as blood fat or blood sugar to reduce the negative influence on the blood fat and the blood sugar. The present disclosure could be widely used for various diseases caused by uric acid crystallization and high uric acid with high uric acid value, for example, such as toe joint gout pain, cardiovascular and cerebrovascular diseases caused by the combination of uric acid molecules and lipoproteins and the like, for specific.

The pH value of the trimethylol aminomethane hydrochloride solution with the pH value of 7.4 is consistent with the pH value in human blood, thus avoiding the influence of strong alkali on the enzyme activity of macromolecular protease in the blood, thereby significantly reducing toxic and side effects.

The indication of trimethylol aminomethane hydrochloride in the prior art is a chemical reagent and could not alkalize urine to treat urinary uric acid stone, as well as to treat hyperuricemia and related diseases thereof.

Adaptation diseases of the trimethylol aminomethane hydrochloride or its solutions in the present disclosure are all of diseases with an over-high uric acid content in serum such as greater than 6.8 mg/dL for men and greater than 6.0 mg/dL for women, including gout, gouty arthritis, cerebral stroke, ischemic heart disease, renal dysfunction, uremia, lithangiuria, gouty nephropathy, kidney stone or chronic kidney disease (CKD), which relate to hyperuricemia. Specific diseases may be, for example, toe joint gout pain, cardiovascular and cerebrovascular diseases caused by the combination of uric acid molecules and lipoproteins and the like.

The trimethylol aminomethane hydrochloride provided by the present disclosure could not alkalize urine and is not a drug to alkalize urine, while is a drug of which molecules combine with uric acid to treat hyperuricemia.

The present disclosure found the new properties of the “trimethylol aminomethane hydrochloride” that can bind with uric acid to form the “uric acid · trimethylol aminomethane hydrochloride” complex, and the “trimethylol aminomethane” that can bind with uric acid to form the “uric acid · trimethylol aminomethane” complex, namely, found new medical use of the two compounds, “trimethylol aminomethane hydrochloride” and “trimethylol aminomethane”. The present disclosure is an invention of medical use, which has novelty, inventive step and presents well effects.

The “trimethylol aminomethane hydrochloride” binds with uric acid to form a “uric acid · trimethylol aminomethane hydrochloride” complex, and the “trimethylol aminomethane” binds with uric acid to form a “uric acid · trimethylol aminomethane” complex, and the principle for these are the three hydroxyl groups of trimethylol aminomethane binding with three oxygen atoms of a uric acid via hydrogen bonds. According to this principle, it will be understood that a pharmaceutically acceptable salt of trimethylol aminomethane could also bind with uric acid, and has use in the manufacture of a medicament for treating or preventing hyperuricemia, gout, acute and chronic arthritis, gouty nephropathy and uric acid kidney stone and related diseases as well.

Based on the action principle of the present disclosure, a composition or conjugate (salt) formed by trimethylol aminomethane with an organic acid or an inorganic acid may be further prepared, which could be used in the manufacture of a medicament for treating or preventing hyperuricemia, gout, acute and chronic arthritis, gouty nephropathy and uric acid kidney stone and related diseases.

For the pharmaceutically acceptable salt of trimethylol aminomethane, the acid forming the salt with trimethylol aminomethane includes an organic acid and an inorganic acid, where the inorganic acid or organic acid includes but is not limited to a lysine, ascorbic acid, acetic acid, formic acid, phosphoric acid, sulfuric acid, maleic acid or butanedisulfonic acid so as to form the salt with trimethylol aminomethane or form a compound via hydrogen bonding.

In the prior art, although it is known that trimethylol aminomethane can alkalize blood and promote the dissolution of uric acid crystals, if trimethylol aminomethane is topically administered through topic skin, a small amount of trimethylol aminomethane enters body parts such as joints and the like, and the pH value of the blood in the joints will be regulated to a certain extent, but the pH value of the blood in the joints would be regulated to a very small extent due to the fact that the blood of a human body is automatically maintained at 7.25-7.35. Thus, the effect of promoting the dissolution of a large amount of uric acid crystals in the joints cannot be achieved, thereby failing to achieve the effect of treating gouty arthritis.

That is, the prior art is limited to the alkalinity of amino in the understanding of the performance of trimethylol aminomethane, which limits and hinders the application of trimethylol aminomethane in the preparation of the topical skin administration external pharmaceutical composition, so in the prior art there is no trimethylol aminomethane topical skin administration external pharmaceutical composition.

According to embodiments of the present disclosure, the new property that trimethylol aminomethane or pharmaceutically acceptable salts thereof can react with uric acid to form a uric acid · trimethylol aminomethane conjugate is discovered, and with the principle of trimethylol aminomethane combining with uric acid, trimethylol aminomethane enters body parts such as joints and the like transdermally, and directly combines with the uric acid therein, thus preventing crystallization of uric acid, thereby achieving the effect of treating gouty arthritis.

Namely, based on the action principle of the present disclosure, trimethylol aminomethane or the pharmaceutically acceptable salts thereof may be applied to the preparation of the external pharmaceutical composition for preventing and treating gout and gouty arthritis via topically transdermal administration. External gels containing trimethylol aminomethane or the pharmaceutically acceptable salts thereof are prepared for preventing and treating gout and gouty arthritis via topically transdermal administration. See Example 5 for specific implementation.

According to the principle that trimethylol aminomethane or the pharmaceutically acceptable salts thereof can combine with the uric acid to form the uric acid · tris trimethylol aminomethane conjugate thereby eliminating the harm of uric acid molecules in the body to the body, dialysis liquids and dialysis powders containing trimethylol aminomethane or the pharmaceutically acceptable salts thereof may be further prepared to prevent and treat related diseases and the like caused by high uric acid in dialysis patients with hyperuricemia.

According to the principle that trimethylol aminomethane or the pharmaceutically acceptable salts thereof can combine with the uric acid to form the uric acid · tris trimethylol aminomethane conjugate thereby eliminating the harm of uric acid molecules in the body to the body, a health-care food, a food, a poultry feedstuff containing trimethylol aminomethane or the pharmaceutically acceptable salts thereof may be further prepared.

According to the principle that trimethylol aminomethane or the salts of the same can combine with the uric acid to form the uric acid · tris trimethylol aminomethane conjugate thereby eliminating the harm of uric acid molecules in the body to the body, a veterinary drug or feedstuff additive containing trimethylol aminomethane or the pharmaceutically acceptable salts thereof may be further prepared to prevent and treat veterinary gout (such as chicken gout) and related diseases.

In specific embodiments, the trimethylol aminomethane hydrochloride solution may be an aqueous solution prepared by dissolving trimethylol aminomethane hydrochloride in water and adding sodium hydroxide to adjust the pH value to between 5.0 and 11; or may be a solution prepared by dissolving the trimethylol aminomethane in water and adding hydrochloric acid to adjust the pH value to 5.0-11, preferably be an aqueous solution with a pH value of 7.4±0.5.

In specific embodiments, injections, oral liquids, tablets, capsules, granules, pills, powders, dialysis liquids, dialysis powders and the like containing trimethylol aminomethane or the trimethylol aminomethane hydrochloride may be prepared; or external pharmaceutical preparations such as gel ointments, sprays and the like via transdermal delivery may be prepared.

In specific embodiments, the action principle of the pharmaceutically acceptable salt of trimethylol aminomethane or the composition formed by trimethylol aminomethane and the organic acid or the inorganic acid being prepared to a drug for preventing or treating hyperuricemia, gout, acute and chronic arthritis, gouty nephropathy and uric acid kidney stone and related diseases is that, the trimethylol aminomethane (hydrochloride) binds with uric acid to form the “uric acid · trimethylol aminomethane (hydrochloride)” complex via hydrogen bonds, thus preventing crystallization of uric acid, dissolving crystals (stones) of the uric acid, and reducing the combination of the uric acid with macromolecular substances such as blood fat or blood sugar to reduce the negative influence on the blood fat and the blood sugar meanwhile.

Adaptation diseases of embodiments of the present disclosure are diseases with an over-high uric acid content such as greater than 420 µmol/L for men and greater than 420 µmol/L for women, including gout, gouty arthritis, cerebral stroke, ischemic heart disease, renal dysfunction, uremia, lithangiuria, gouty nephropathy, kidney stone or chronic kidney disease (CKD), which are related to hyperuricemia, for example, toe joint gout pain, cardiovascular and cerebrovascular diseases and the like caused by the combination of uric acid molecules and lipoproteins.

In relative to the three existing action principles for treating hyperuricemia and gout, i.e. a. reducing intake; b. inhibiting endogenous synthesis; and c. promoting renal excretion, the action principle provided by the present disclosure may be taken as the fourth action principle, in which the drug molecule directly combines with uric acid thereby eliminating the harm of uric acid. Accordingly, the treatment solution of the present disclosure for gout may be referred to as the fourth scheme for treating gout.

In the prior art, the method of alkalizing urine by using trimethylol aminomethane to treat uric acid crystallization in the urinary system and other methods of inhibiting endogenous synthesis or promoting renal excretion, only solve the problems of uric acid amount and uric acid crystallization, but cannot solve the problems of the combination of uric acid with blood fat, blood sugar and other macromolecular substances and the influence on the same.

The new medicine trimethylol aminomethane or the pharmaceutically acceptable salt thereof provided by embodiments of the present application not only can solve the problem of crystallization of uric acid, but also can solve the problem of the influence of uric acid on blood fat and blood sugar.

The present application has the innovative advantages of clear action mechanism, strong pertinence, good curative effect and small side effect, and has wide application range, high safety and good social and economic benefits.

As used herein, the term “composition” is intended to encompass a product comprising the specified ingredients in the specified amounts, as well as any product resulting directly or indirectly from the combination of the specified ingredients in the specified amounts. The term in relation to a pharmaceutical composition is intended to encompass a product comprising an active ingredient and an inert ingredient constituting a carrier, as well as any product resulting, directly or indirectly, from the combination, complexation or polymerization of any two or more ingredients, or from other types of reactions or interactions, e.g. resulting in the decomposition of one or more ingredients. Thus, the pharmaceutical compositions of the present invention encompass any composition prepared by mixing the compounds of the present invention with pharmaceutically acceptable excipients.

Unless defined otherwise, all technical and scientific terms used herein have the same meaning as commonly understood by one ordinary skilled in the art to which this invention belongs. In case of conflict, that present specification, include definitions, will control. Throughout the specification and claims, the word “comprise,” or variations such as “comprises” or “comprising,” will be understood to imply the inclusion of a stated integer or group of integers but not the exclusion of any other integer or group of integers. Unless the context requires otherwise, nouns that are not limited by numerals include both singular and plural referents. Any examples following the terms “such as” or “such as” are not meant to be exhaustive or limiting.

### EXAMPLE

### Example 1: The Trimethylol Aminomethane Hydrochloride Binding With Uric Acid to Form the “Uric Acid · Trimethylol Aminomethane (Hydrochloride) Complex” and Study on the “Uric Acid · Trimethylol Aminomethane (Hydrochloride) Complex”

1.1 300 mg of uric acid were weighted and added into 100 ml of 0.6 mol/L (pH 7.4) of a trimethylol aminomethane hydrochloride solution, stirring for 10 minutes, and the uric acid was gradually dissolved, and the solution became clear; with stirring continually, then milky flocculent precipitate was generate; and with vacuum filtration and drying, 480 mg uric acid - trimethylol aminomethane complex in white lump were obtained. As the presence of “trimethylol aminomethane” and “trimethylol aminomethane hydrochloride” in the trimethylol aminomethane hydrochloride solution with pH 7.4, the products included “uric acid · trimethylol aminomethane” and “uric acid · trimethylol aminomethane hydrochloride”.

The reaction equations are as follows:

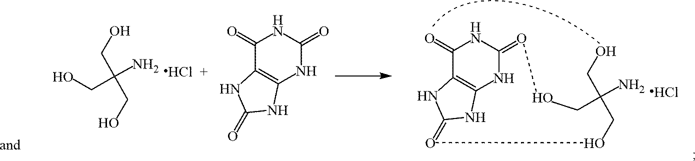

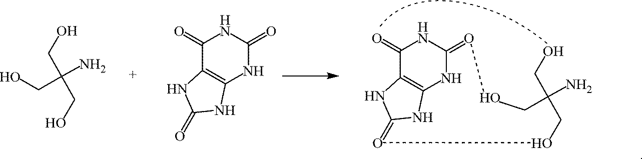

1.2 300 mg of uric acid were weighted and added into 100 ml of 0.6 mol/L of trimethylol aminomethane in aqueous solution, stirring for 10 minutes, and the uric acid was gradually dissolved, and the solution became clear; with stirring continually, then milky flocculent precipitate was generate; and with vacuum filtration and drying, uric acid - trimethylol aminomethane complex in white lump was obtained.

The reaction equation is as follows:

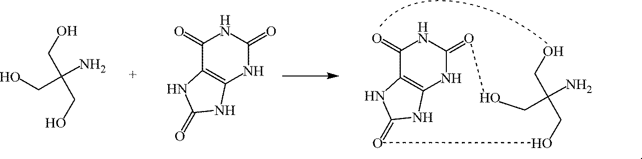

### 1.3 Properties of the Uric Acid · Trimethylol Aminomethane (Hydrochloride) Complex

### 1.3.1 Solubility

1.3.1.1 56 mg of the uric acid - trimethylol aminomethane complex in white lump were added into 100 ml of water, stirring for 1 hour, and the white lumps were completely dissolved, and the solution was clear and did not recrystallize. However, if the uric acid - trimethylol aminomethane complex was added further, the solution would be supersaturated and some of the complex would not be dissolved.

It indicates that the solubility of the uric acid - trimethylol aminomethane complex was about 8 times that of uric acid.

1.3.1.2 21 mg of uric acid were weighed and added to 100 ml of 8 mM (pH 7.4) of a trimethylol aminomethane hydrochloride solution, stirring for 1 hour, and the uric acid was completely dissolved, and the solution became clear; with stirring continually, and then no milky precipitate was generated, and the solution remained clear.

It indicates that the uric acid - trimethylol aminomethane complex formed in blood did not precipitate as crystals after treatment with trimethylol aminomethane hydrochloride solution even for uric acid up to 1260 µmol/L.

### 1.3.2 Melting Point

The melting point of the uric acid - trimethylol aminomethane complex is > 270° C. (the crystal turned copper yellow during heating).

### 1.3.3 Ultraviolet Detection

There are absorption peaks at 233 nm and 293 nm, and the maximum absorption peak is at 293 nm.

### 1.3.4 Infrared Detection Spectrum

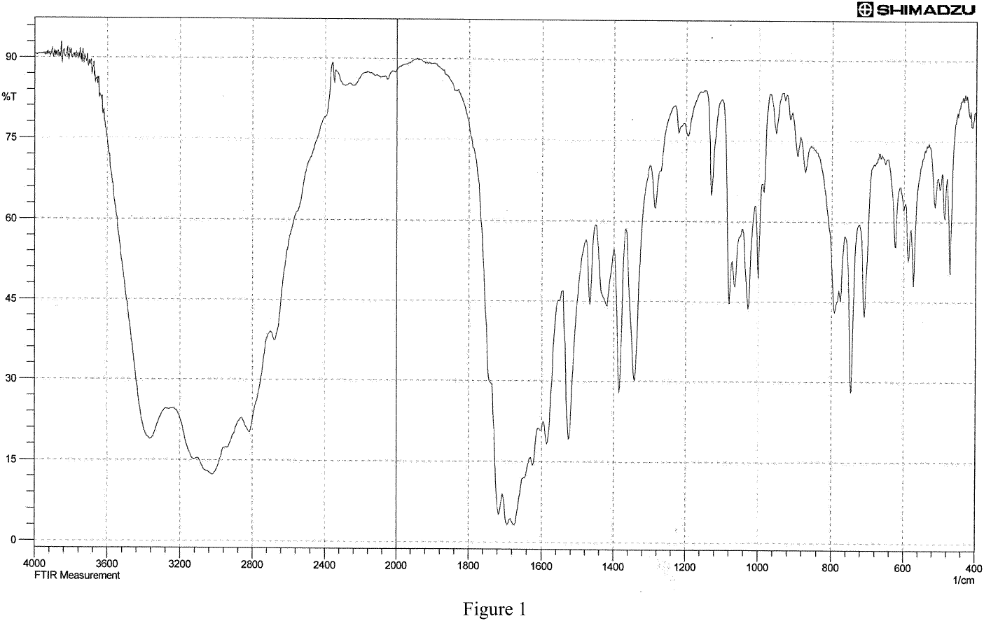

See FIG. 1.

### Example 2: Experiment of Dissolving Uric Acid Crystals Simulating Human Administration

Experimental materials were as follows: a. uric acid (crystal); b. 0.3 mol/L or 0.6 mol/L of a trimethylol aminomethane hydrochloride solution with pH 7.4±0.5; and c. water for injection.

### In Vitro Simulation Test

Background: An assumption was set that a 60 kg hyperuricemic patient with a total blood volume of 4500 ml has 7 mg of uric acid dissolved in 100 ml of blood and 3 mg of uric acid crystals in 100 ml of blood. In this Example, 0.3 mol/L or 0.6 mol/L of a trimethylol aminomethane hydrochloride solution with pH 7.4±0.5 was used for treatment, and the administration amount is 0.5 ml/kg/dose to 3 ml/kg/dose.

### Scheme

### For 0.3 mol/L of the Trimethylol Aminomethane Hydrochloride With the Administration Amount of 0.5ml/kg/dose

10 mg of uric acid, 100 ml of water and 0.66 ml of 0.3 mol/L of the trimethylol aminomethane hydrochloride solution with pH 7.4±0.5 were taken and the mixture was placed into a shaker for shaking at 36.5° C. for 4 hours with 60 rpm, and the result is that the solution was clear and no longer crystallized.

### For 0.3 mol/L of the Trimethylol Aminomethane Hydrochloride With the Administration Amount of 1.0 ml/kg/dose

10 mg of uric acid, 100 ml of water and 1.33 ml of 0.3 mol/L of the trimethylol aminomethane hydrochloride solution with pH 7.4±0.5 were taken and the mixture was placed into a shaker for shaking at 36.5° C. for 2 hours with 60 rpm, and the result is that the solution was clear and no longer crystallized.

### For 0.3 mol/L of the Trimethylol Aminomethane Hydrochloride With the Administration Amount of 1.5 ml/kg/dose

10 mg of uric acid, 100 ml of water and 1.99 ml of 0.3 mol/L of the trimethylol aminomethane hydrochloride solution with pH 7.4±0.5 were taken and the mixture was placed into a shaker for shaking at 36.5° C. for 2 hours with 60 rpm, and the result is that the solution was clear and no longer crystallized.

### For 0.3 mol/L of the Trimethylol Aminomethane Hydrochloride With the Administration Amount of 2.0 ml/kg/dose

10 mg of uric acid, 100 ml of water and 2.66 ml of 0.3 mol/L of the trimethylol aminomethane hydrochloride solution with pH 7.4±0.5 were taken and the mixture was placed into a shaker for shaking at 36.5° C. for 30 min with 60 rpm, and the result is that the solution was clear and no longer crystallized.

### For 0.3 mol/L of the Trimethylol Aminomethane Hydrochloride With the Administration Amount of 3.0 ml/kg/dose

10 mg of uric acid, 100 ml of water and 4.0 ml of 0.3 mol/L of the trimethylol aminomethane hydrochloride solution with pH 7.4±0.5 were taken and the mixture was placed into a shaker for shaking at 36.5° C. for 30 min with 60 rpm, and the result is that the solution was clear and no longer crystallized.

### For 0.6 mol/L of the Trimethylol Aminomethane Hydrochloride With the Administration Amount of 0.5 ml/kg/dose

15 mg of uric acid, 100 ml of water and 0.66 ml of 0.6 mol/L of the trimethylol aminomethane hydrochloride solution with pH 7.4±0.5 were taken and the mixture was placed into a shaker for shaking at 36.5° C. for 4 h with 60 rpm, and the result is that the solution was clear and no longer crystallized.

### For 0.6 mol/L of the Trimethylol Aminomethane Hydrochloride With the Administration Amount of 1.0 ml/kg/dose

15 mg of uric acid, 100 ml of water and 1.33 ml of 0.6 mol/L of the trimethylol aminomethane hydrochloride solution with pH 7.4±0.5 were taken and the mixture was placed into a shaker for shaking at 36.5° C. for 2 h with 60 rpm, and the result is that the solution was clear and no longer crystallized.

### For 0.6 mol/L of the Trimethylol Aminomethane Hydrochloride With the Administration Amount of 1.5 ml/kg/dose

15 mg of uric acid, 100 ml of water and 1.99 ml of 0.6 mol/L of the trimethylol aminomethane hydrochloride solution with pH 7.4±0.5 were taken and the mixture was placed into a shaker for shaking at 36.5° C. for 30 min with 60 rpm, and the result is that the solution was clear and no longer crystallized.

The results of dissolving uric acid crystals simulating administration in vivo in the trimethylol aminomethane hydrochloride solution are shown in Table 1 below.

TABLE 1

results of dissolving uric acid crystals in the trimethylol aminomethane hydrochloride solution

| | uric acid amount assumed in 100 ml of blood | | administration amount | | Specification of drug liquid | Dissolving time | results |
|---|---|---|---|---|---|---|---|
| | dissolved | crystalized | ml/kg | ml | | Hour | |
| 1 | 7 mg | 3 mg | 0.5 | 30 | 0.3 mol/L | 4 | Clear |
| 2 | 7 mg | 3 mg | 1.0 | 60 | 0.3 mol/L | 2 | Clear |
| 3 | 7 mg | 3 mg | 1.5 | 90 | 0.3 mol/L | 2 | Clear |
| 4 | 7 mg | 3 mg | 2.0 | 120 | 0.3 mol/L | 0.5 | Clear |
| 5 | 7 mg | 3 mg | 3.0 | 180 | 0.3 mol/L | 0.5 | Clear |
| 6 | 7 mg | 8 mg | 0.5 | 30 | 0.6 mol/L | 4 | Clear |
| 7 | 7 mg | 8 mg | 1.0 | 60 | 0.6 mol/L | 2 | Clear |
| 8 | 7 mg | 8 mg | 1.5 | 90 | 0.6 mol/L | 0.5 | Clear |

The above tests show that for hyperuricemic patients, the trimethylol aminomethane hydrochloride solution (pH 7.4 ± 0.5) of the present disclosure at a concentration of 0.3 mol/L or 0.6 mol/L can significantly dissolve uric acid crystals in blood and prevent them from crystallizing subsequently.

### Example 3: Solubility Test of Sodium Urate Crystals in the Trimethylol Aminomethane Hydrochloride Solution

Experimental example: 35 ml of 0.3 mol/L of the trimethylol aminomethane hydrochloride solution with pH 7.4±0.5 were added with 50 mg of sodium urate, and the mixture was placed into a shaker for shaking at 36.5° C. for 4 hours with 60 rpm, and the result is that the sodium urate crystals were dissolved completely and the solution was clear.

Comparative example: 35 ml of 0.3 mol/L of purified water were added with 50 mg of sodium urate, and the mixture was placed into a shaker for shaking at 36.5° C. for 4 hours with 60 rpm, and the result is that the sodium urate was mostly undissolved and the solution was cloudy.

The test results of the above indicate that the trimethylol aminomethane hydrochloride solution can dissolve sodium urate crystals significantly.

### Example 4: The Trimethylol Aminomethane Hydrochloride Solution for Preventing and Treating Crystallization of Uric Acid

In this example, the prevention of formation of uric acid crystals in rat kidneys and the treatment of crystallization of uric acid in rat kidneys by a trimethylol aminomethane hydrochloride solution (hereinafter referred to as Changsheng No. 1) were investigated.

Experimental material information is as follows.

Sample for test: a trimethylol aminomethane hydrochloride solution with pH 7.4±0.5, transparent and clear liquid, referred to as Changsheng No.1.

Modeling drug 1: potassium oxonate, purchased from Beijing Solaibao Technology Co., Ltd.

Modeling drug 2: hypoxanthine, purchased from Sigma-Aldrich China.

An appropriate amount of potassium oxonate or hypoxanthine was accurately weighed and prepared into suspension with 0.5% CMC-Na. The suspension was stored at room temperature after sub-packaging, and used within 12 h. The concentrations of the suspensions were both 60 mg/mL.

Reagents and kits: a uric acid assay kit with batch number of 20200728, purchased from Nanjing Jiancheng Technology Co., Ltd.

Instruments: ECC2201 electronic balance, ECC5201 electronic balance and DNA103A electronic balance, all purchased from Nanjing Bonita Scientific Instruments Co., Ltd; and R-6100 microplate reader, purchased from Shenzhen Leidu.

Experimental animals: SD rats, male, SPF grade, purchased from Speyford (Beijing) Biotechnology Co., Ltd., with Animal Production License No.: SYXK (J) 2019-0010 and Certificate No.:110324201101852.

The method of animal use in the Example was approved by the Institutional Animal Care and Use Committee (IACUC).

Experimental steps were as follows.

Thirty-five male SD rats, weighing 240 - 260 g, were randomly divided into 5 groups according to body weight, namely a normal control group, model control group and three dose groups treated by Changsheng No.1 with 0.85 g/kg/day, 1.70 g/kg/day and 3.40 g/kg/day individually, with 7 rats in each group.

Except for the normal control group, the other groups were intraperitoneally injected with 300 mg/kg of potassium oteronate and intragastrically perfused with 300 mg/kg of hypoxanthine, and the volume of injection and intragastrical perfusion were 5 ml/kg respectively. The normal control group was administered with the same volume of 0.5% CMC-Na, and the model was established at 0.5 h after the second administration every day with once a day and for five consecutive days. Changsheng No. 1 was administered by gavage at a dose volume of 10 ml/kg, twice a day (dose halved/time), with an interval of about 4 h, for five consecutive days. The normal and model control groups were administered with the same volume of 0.5% CMC-Na by gastric perfusion.

On the 4th day of the test, the rats were fasted overnight (about 16 h). On the 5th day of the test, the rats were weighed and anesthetized by intraperitoneal injection of 60 mg/kg sodium pentobarbital, with an injection volume of 2 ml/kg. About 0.7 mL of blood was collected from the orbit and then the kidney was removed. The whole blood was centrifuged at 6000 r/min for 10 min, the supernatant was taken and stored at -20° C. for keeping the blood uric acid content to be determined. The kidney was weighed, opened to observe the deposition of uric acid crystals in white radial pattern, and stored at -70° C. for future use.

Uric acid content determination: After the serum was rapidly remelted, the uric acid content was determined according to the method specified in the instructions for use, and the serum uric acid content was calculated.

Data processing: The data were expressed as mean ± standard deviation (x ± SD) and analyzed with SPSS 25.0 software. One-Way ANOVA was used for comparison among multiple groups. LSD test was used for homogeneity of variance, where P <0.05 was considered as statistically significant.

Test results are as follows.

### 4.1 Prevention of the Formation of Uric Acid Crystals and Treatment of Crystallization of Uric Acid Crystal in the Kidneys

### 4.1.1 Effects on Renal Uric Acid Crystal Deposition

The kidneys of rats in the normal control group were smaller and found no radial white line. In the model control group, 14 kidneys (7 rabbits ×2 kidneys/rabbit) were observed with obvious radial white lines. In the group treated by 0.85 g/kg of Changsheng No.1, 11/14 kidneys were observed with obvious radial white lines. In the group treated by 1.7 g/kg of Changsheng No.1, 5/14 kidneys were observed with obvious radial white lines. In the group treated by 3.40 g/kg of Changsheng No.1, none of 14 kidneys were observed with obvious radial white lines, which presented no difference from that of the normal control group.

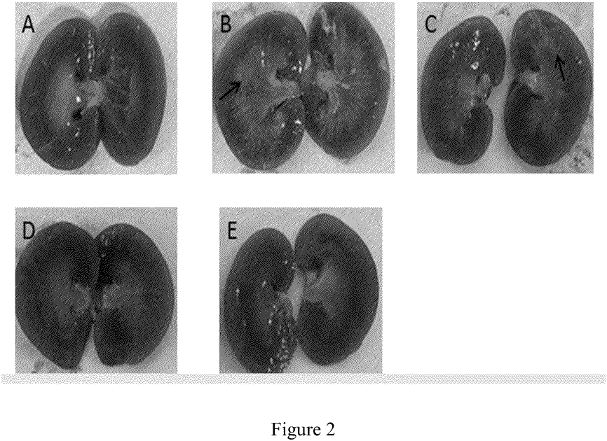

The effect of Changsheng No.1 on the deposition of uric acid crystals in the kidney is shown in FIG. 2, where an arrow indicates the crystal deposition of uric acid in the kidney, and A is a normal control group, B is a model control group, C is a group treated with 0.85 g/kg of Changsheng No. 1, D is a group treated with 1.70 g/kg of Changsheng No. 1, and E is a group treated with 3.40 g/kg of Changsheng No. 1.

### 4.1.2 Effect on Renal Coefficient

Compared with the normal control group, the renal coefficient of the model control group was significantly increased (P<0.001). This was due to the deposition of uric acid crystals in the kidney, indicating successful modeling. The renal coefficients of the group treated with 0.85 g/kg of Changsheng No. 1 and 1.70 g/kg of Changsheng No. 1 were also significantly higher than that of the normal control group (P<0.01 and P<0.05 respectively), and the pharmacodynamic effect was slightly weak. There was no significant difference between the group treated with 3.40 g/kg of Changsheng No. 1 and the normal control group.

Compared with the model control group, the renal coefficients of the group treated with 1.70 g/kg of Changsheng No. 1 and 3.40 g/kg of Changsheng No. 1 were significantly reduced (P<0.05 and P<0.001 respectively), as shown in Table 2.

TABLE 2

Effects of Changsheng No. 1 on kidney weight and renal coefficient of rat

| Group | Dose (g/kg) | Weight (g) | kidney weight (g) | renal coefficient (g/100g·bw) |
|---|---|---|---|---|
| normal control group | - | 272.1±14.3 | 2.139±0.185 | 0.785±0.039 |
| model control group | - | 260.7±11.0 | 3.022±0.411 | 1.157±0.129 |
| Changsheng No. 1 | 0.85 | 255.8±7.6 | 2.636±0.491 | 1.034±0.217 |
| Changsheng No. 1 | 1.70 | 264.4±9.0 | 2.507±0.461 | 0.953±0.206 |
| Changsheng No. 1 | 3.40 | 266.6±9.2 | 2.207±0.114 | 0.829±0.052 |

Note: Compared with the model control group, P<0.05 and P<0.001; compared with the normal control group, P<0.05, P<0.01 and P<0.001; n represents sample numbers.

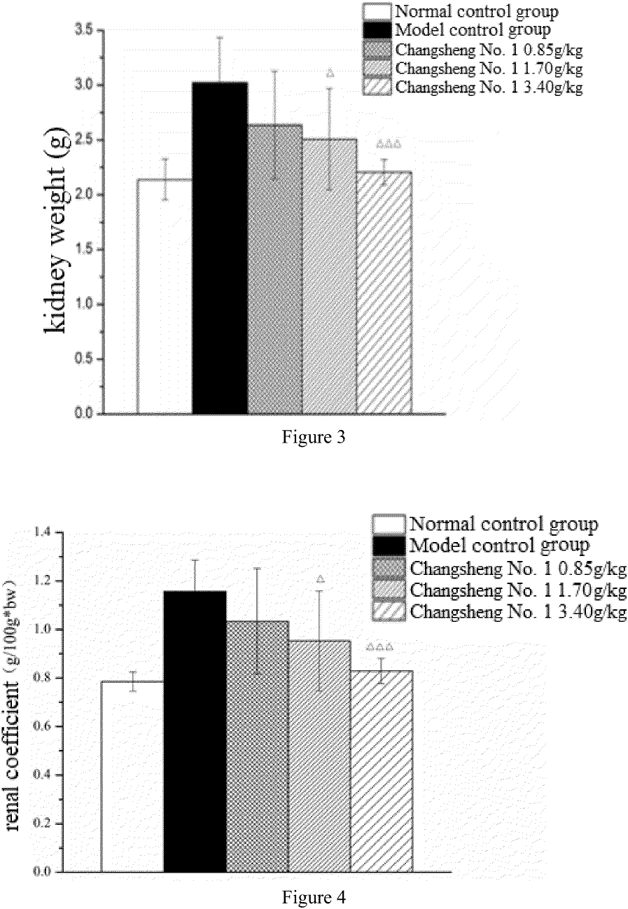

The effects of Changsheng No. 1 on kidney weight are shown in FIG. 3, where compared with the model control group, P<0.05 and P<0.001.

The effects of Changsheng No. 1 on renal coefficient are shown in FIG. 4, where compared with the model control group, P<0.05 and P<0.001.

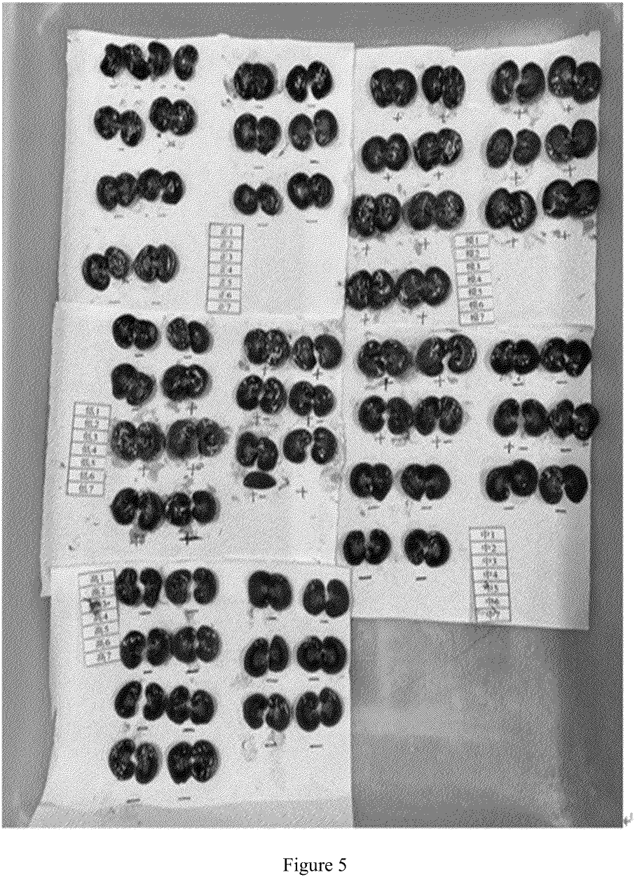

4.1.3 FIG. 5 shows observed results of uric acid crystallization in rat kidneys, where the kidney in the group treated with 3.40 g/kg of Changsheng No. 1 presents no difference from that of the normal control group.

### 4.2 Effect on Blood Uric Acid Content

As shown in Table 3, compared with the normal control group, the blood uric acid content of the model control group and the groups treated with 0.85 g/kg, 1.70 g/kg and 3.40 g/kg of Changsheng No. 1 were significantly increased (P<0.05, P<0.05 and P<0.05 respectively). Compared with the model control group, all of the groups treated with Changsheng No. 1 presented the effect of improvement.

TABLE 3

Effects of Changsheng No. 1 on blood uric acid content of rat

| Group | Dose (g/kg) | serum uric acid (µmol/L) |
|---|---|---|
| normal control group | - | 224.37±13.33 |
| model control group | - | 1018.46±279.00 |
| Changsheng No. 1 | 0.85 | 976.91±172.29 |
| Changsheng No. 1 | 1.70 | 838.42±3.19 |
| Changsheng No. 1 | 3.40 | 965.17±215.90 |

Note: Compared with the normal control group, P<0.01 and P<0.001; n represents sample numbers.

The above experiments showed that the trimethylol aminomethane hydrochloride solution at pH 7.4 (i.e. Changsheng No.1) dose-dependently reduced uric acid crystal deposition in the kidney. The observed color and size of the kidneys in the group treated with 3.40 g/kg of Changsheng No.1 were the same as that in the normal control group, and there was no statistical difference in kidney coefficient between the normal group and the group treated with 3.40 g/kg of Changsheng No.1, indicating that Changsheng No.1 could prevent and treat renal inflammation and swelling caused by high uric acid, and could prevent and treat uric acid crystal deposition in kidney.

### Example 5

### 5.1 Preparation of Gels of the Trimethylol Aminomethane Hydrochloride

In a 1 L beaker, 700 ml of water, 12 g of Carbomer and 156 g of trimethylol aminomethane hydrochloride were added and stirred for 1 h, and pH of the mixture was adjusted to 7.4 with sodium hydroxide solution, and then maked up to 1 L with water, to obtain trimethylol aminomethane hydrochloride gel. The trimethylol aminomethane hydrochloride gel was sub-packaged into 50 ml plastic bottle to obtain the finished product of the gels of the trimethylol aminomethane hydrochloride for treating gout.

### 5.2 Preparation of Gels of the Trimethylol Aminomethane

5.2.1 In a 1 L beaker, 500 ml of water and 12 g of Carbomer were added and stirred for 1 h, and then 15 g of triethanolamine were added and stirred for 1 h, and the pH value was measured as 7.2.

5.2.2 In a 1 L beaker, 220 ml of water and 121 g of trimethylol aminomethane were added and stirred for 10 min, then for 1 h.

5.2.3 The mixture in 5.2.2 was poured into the mixture in 5.2.1, and maked up to 1 L with water and stirred for 1 h, and the pH value was measured as 11.1 to obtain trimethylol aminomethane gel.

The trimethylol aminomethane gel was sub-packaged into 50 ml plastic bottle to obtain the finished product of the gels of the trimethylol aminomethane for treating gout.

### 5.3 Preparation of Blank Excipient in Relative to Gels of the Trimethylol Aminomethane

In a 1 L beaker, 500 ml of water and 12 g of Carbomer were added and stirred for 1 h, and then 15 g of triethanolamine were added and stirred for 1 h, followed by making up to 1 L with water and the pH value was measured as 7.2, to obtain the blank excipient in relative to gels of the trimethylol aminomethane.

### 5.4 Curative Effect of Gels of the Trimethylol Aminomethane on Gouty Arthritis in Animals

### Establishment of Gouty Arthritis Rat Model

Thirty healthy male SD rats weighing 180 - 220 g were selected and randomly divided into 3 groups with 10 rats in each group, namely the normal control group (no modeling), the model group and the treatment group (treated with the gels of the trimethylol aminomethane).

Referring to the classic method of establishing acute gouty arthritis model by Coderre et al., the rats in the model group and the treatment group were fixed after anesthesia, and the ankle joint of the right hind leg was disinfected with iodophor, and No. 6 injection needle was inserted into the medial side of the tibial tendon from 45° direction on the dorsal side of the ankle joint of the right hind leg, and 0.2 mL of 10% sodium urate solution was injected into each rat after feeling empty. The gouty arthritis model was prepared by taking the redness and swelling of the ankle joint and paw as the success standard of modeling.

From the day after modeling, the treatment group began to administer drugs.

For the treatment group: the gels of the trimethylol aminomethane (i.e. the product of 5.2) were smeared and dressed with a thickness of about 0.2 cm on the ankle skin below 0.5 cm above the upper end of the ankle joint of the right hind limb, 6 times/day for 2 consecutive days.

For the model group: the blank excipient without the trimethylol aminomethane (i.e. the product of 5.3) was smeared, and the operation was the same as that of treatment group.

The circumference of the same part of the ankle joint of the right hind leg of the rats in the model group and the treatment group was measured by ligature method before and after modeling and after the last administration (2 hours), and the joint swelling degree of each rat was calculated.

The joint swelling degree = (circumference after the last administration - circumference before modeling) /circumference before modeling. During the experiment, the skin symptoms of rats in each group in the administration area were observed, including erythema and rash.

After the last administration, the rats were killed after the measurement of the joint swelling degree and the observation of local symptoms after administration, and the right hind limb ankle joint was taken as the center for cutting at 0.5 cm above and below it. The tested joint and surrounding soft tissues were removed and the ankle joint capsule was cut on the ice tray. The joint capsule, synovium and other tissues were rapidly cut off and weighed, diluted with normal saline according to the weight ratio of 1: 9, homogenized, and centrifuged at 12000 rpm for 30 min at 4° C. The supernatant was sub-packaged and stored in a refrigerator at -70° C. for testing.

The observed results during the experiment are that, the rats in each group had no skin symptoms in the administration area, such as erythema and rash.

The levels of C-reactive protein (CRP) and tumor necrosis factor (TNF) in j oint extracts were measured. The CRP level was detected by immune turbidimetry. The level of TNF-a was detected by double antibody sandwich enzyme-linked immunosorbent assay (ELISA). The contents of CRP and TNF-α in the j oint extracts were determined according to the instructions of the kit.

The determination results of the joint swelling degree and inflammatory factor level of joint extracts of rats in each group are shown in the table below.

Determination results

| Group | joint swelling degree % | Mean value of CRP (mg/L) | Mean value of TNF-a (Pg/ml) |
|---|---|---|---|
| Normal group | --- | 2.82 | 18.57 |
| Model group | 26.7±0.15 | 8.67 | 39.22 |
| Treatment group | 3.2±0.09 | 3.69 | 20.36 |

It can be seen from the above table that the joint swelling degree and the levels of CRP and TNF-α in joint extracts in the treatment group were significantly lower than those in the model group, indicating that the gels of trimethylol aminomethane had significant therapeutic effect on the symptoms and inflammatory factors of gouty arthritis rats.

### 5.5 Efficacy of the Gels of Trimethylol Aminomethane Hydrochloride and the Gels of Trimethylol Aminomethane in the Treatment of Patients with Gouty Toe Redness and Pain

The products of the gels of trimethylol aminomethane hydrochloride and the gels of trimethylol aminomethane prepared in 5.1 and 5.2 respectively were provided to the classmates or relatives and friends of the personnel in the research group with gout in the form of free trial, and they tried the products under the condition of full notification and consent to ensure safety.

Case 1 was Zhao XX, 58 years old, with a 5-year gout medical history. According to the patient’s description, when she was examined in the hospital, it was confirmed that she was suffered from hyperuricemia gout, and gout often attacks. The main attack site is the toe joint, when it attacked, the big toe was burned like, unbearable, accompanied by redness and swelling.

The gels of trimethylol aminomethane hydrochloride prepared in 5.1 was smeared with a thickness of about 0.2 cm, and the coating was dressed with an external application bag. The painful part was smeared once an hour to keep it moist. After 40 minutes, the pain began to subside, and after 3 hours, the pain disappeared. The redness and swelling began to be relieved from about 2 hours, and disappeared completely about 20 hours later.

Case 2 was Li XX, 54 years old, with a 6-year gout medical history. According to the patient’s description, he had gout with high uric acid. He had used a medical hall herbal gout gel before, with little effect. He said that gout often attacks at night, and the main attack site is two toe joints and instep, when night sudden attack, it likes a needle sticking the big toe, and accompanied by acupuncture, knife-like sharp pain, piercing pain, and accompanied by redness and swelling.

The gels of trimethylol aminomethane prepared in 5.2 was smeared with a thickness of about 0.2 cm, and the coating was dressed with an external application bag. The painful part was smeared once an hour to keep it moist. After 0.5 hour, the pain began to subside, and after 4 hours, the pain disappeared. The redness and swelling began to be relieved from about 3 hours, and disappeared completely about 24 hours later.

In the above Examples, the trimethylol aminomethane hydroxymethyl and trimethylol aminomethane have short onset time (i.e., fast onset) for gouty arthritis pain and good detumescence effect. In particular, the speed of pain relief was beyond the expectation of patients.

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments”, “in one embodiment”, “in an embodiment”, “in another example”, “in an example”, “in a specific example” or “in some examples”, in various places throughout this specification are not necessarily referring to the same embodiment or example of the present disclosure. Furthermore, the particular features, structures, materials, or characteristics may be combined in any suitable manner in one or more embodiments or examples. Furthermore, different embodiments or examples and features of different embodiments or examples described in this specification may be combined and incorporated by those skilled in the art, where not mutually contradictory.

Although explanatory embodiments have been shown and described, it would be appreciated by those skilled in the art that the above embodiments cannot be construed to limit the present disclosure, and changes, alternatives, and modifications can be made in the embodiments in the scope of the present disclosure.

What is claimed is:

1-18. (canceled)

19. A method of prevention or treatment of a disease caused by the combination (crystallization) between uric acid molecules or between the uric acid molecule and a macromolecular substance, comprising administering a therapeutically effective amount of a pharmaceutically acceptable salt of trimethylol aminomethane or a composition comprising the same to a subject in need thereof, wherein the prevention or treatment is achieved by eliminating a harmful effect of the uric acid via forming a uric acid - trimethylol aminomethane complex via bonding between trimethylol aminomethane and a uric acid, 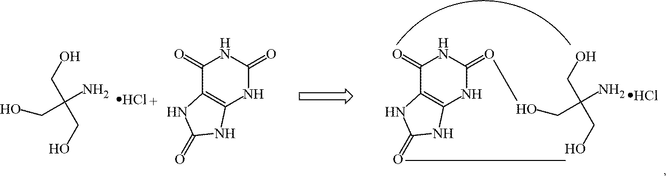
optionally, the disease is gout or gouty nephropathy, and the macromolecular substance is low density lipoprotein.

20. The method according to claim 19, wherein an acid forming the salt with trimethylol aminomethane comprises an organic acid and an inorganic acid, the inorganic acid or organic acid is selected from a group consisting of a hydrochloric acid, lysine, ascorbic acid, acetic acid, formic acid, phosphoric acid, sulfuric acid, maleic acid or butanedisulfonic acid, so as to form the salt with trimethylol aminomethane or form a compound via hydrogen bonding.

21. The method according to claim 19, wherein the disease is gout, gouty nephropathy, gouty arthritis, uremia, lithangiuria, urate nephropathy, kidney stone or chronic kidney disease (CKD).

22. The method according to claim 19, wherein the therapeutically effective amount of the pharmaceutically acceptable salt of trimethylol aminomethane or the composition comprising the same exhibits the following effects: preventing crystallization of a uric acid, dissolving a crystal of the uric acid and reducing a combination of the uric acid with a macromolecular substance such as blood fat or blood sugar so as to reduce the influence on the blood fat and the blood sugar to eliminate a harmful effect of the uric acid or realize a function for reducing the content of the uric acid.

23. The method according to claim 19, wherein the composition comprises a therapeutically effective amount of the pharmaceutically acceptable salt of trimethylol aminomethane and a food acceptable or pharmaceutically acceptable excipients.

24. The method according to claim 23, wherein the composition is selected from a pharmaceutical composition, a feedstuff composition, a food composition or a health-care composition, optionally the pharmaceutical composition is a human medicament or a poultry medicament.

25. The method according to claim 19, wherein the subject comprises a mammal or a non-mammal,
preferably, the mammal is human,
preferably, the non-mammal is poultry, more preferably, the poultry is a goose or chicken.

26. The method according to claim 19, wherein the pharmaceutically acceptable salt of trimethylol aminomethane or the composition comprising the same is formulated in the form of injections, oral liquids, tablets, capsules, dialysis liquids, granules, pills, powders, gel ointments or sprays, and is administered to the subject via infusion, orally, smearing, dressing or dialysis.

27. The method according to claim 19, wherein the amount for administration is 0.1 g/kg to 10 g/kg body weight per day, preferably 1.7 g/kg body weight per day, more preferably 3.4 g/kg body weight per day.

28. The method according to claim 19, wherein when the composition is in the form of solutions, the composition is formulated as an aqueous solution with a pH value of 5.0 to 11.0, preferably an aqueous solution with a pH value of 7.4 ± 0.5.

29. A method of prevention or treatment of gout or gouty arthritis, comprising administering a therapeutically effective amount of trimethylol aminomethane or a pharmaceutically acceptable salt thereof, wherein the treatment or prevention are via topical transdermal administration with the form of topical external transdermal medicament.

30. The method according to claim 29, wherein the topical external transdermal medicament is in the form of creams, ointments, gels, transdermal preparations, foams, sprays, lotions, solutions, emulsions or suspensions, preferably gels.

31. The method according to claim 19, wherein the disease is hyperuricemia, gout or a disease caused by a high content of a uric acid.

32-33. (canceled)

34. The method according to claim 31, wherein the prevention or treatment of hyperuricemia, gout or gouty arthritis in poultry, comprises administering a therapeutically effective amount of trimethylol aminomethane or a composition comprising the same to a poultry subject in need thereof, wherein the prevention or treatment is achieved by forming a uric acid - trimethylol aminomethane complex via bonding between trimethylol aminomethane and a uric acid, 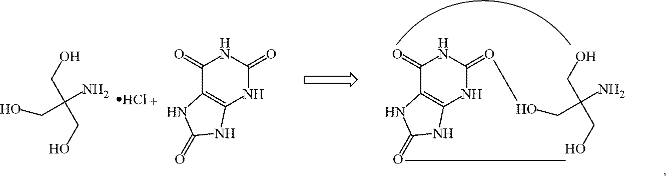
.

35. (canceled)
